(19) United States
(12) Patent Application Publication
(10) Pub. No.: US 2023/0173255 A1
(43) Pub. Date: Jun. 8, 2023

(54) HIGH-THROUGHPUT POROUS SUBSTRATE ELECTROPORATION DEVICES AND METHODS

(71) Applicant: NUtech Ventures, Lincoln, NE (US)

(72) Inventors: Justin Brooks, Lincoln, NE (US); Ruiguo Yang, Lincoln, NE (US); Ikhlaas Mungloo, Lincoln, NE (US)

(21) Appl. No.: 17/933,947

(22) Filed: Sep. 21, 2022

Related U.S. Application Data
(60) Provisional application No. 63/246,421, filed on Sep. 21, 2021.

(51) Int. Cl. A61N 1/04

(52) U.S. Cl. CPC A61N 1/0408; A61N 1/327

(57) ABSTRACT

Electroporation devices and methods of making the same. An electroporation device includes a plurality of independently controllable or addressable electrode pairs, a plurality of reaction chambers, each reaction chamber including a first chamber, a second chamber and a porous substrate separating the first chamber from the second chamber, and each reaction chambers being disposed between one of the plurality of independently controllable or addressable electrode pairs, a plurality of first microfluidic channels configured to deliver a cargo solution from a cargo inlet port to the plurality of first chambers, and a plurality of second microfluidic channels configured to deliver a cell culture from a cell inlet port to the plurality of second chambers. In operation, application of a voltage to an electrode pair permeabilizes the membranes of the cells adhered to the porous substrate in the reaction chamber disposed between the electrode pair.

### CROSS REFERENCE TO RELATED APPLICATION

This patent application claims priority to U.S. Provisional Patent Application No. 63/246,421, entitled “HIGH-THROUGHPUT POROUS SUBSTRATE ELECTROPORATION DEVICES AND METHODS,” filed Sep. 21, 2021, which is incorporated herein by reference in its entirety.

### STATEMENT REGARDING FEDERALLY SPONSORED RESEARCH OR DEVELOPMENT

This invention was made with Government support under project numbers 1826135 and 1936065 awarded by the National Science Foundation and under P20 GM113126 and P30 GM127200 awarded by the National Institutes of Health. The Government has certain rights in this invention.

### BACKGROUND

The ability to intracellularly deliver and extract molecules while preserving cell health is paramount for biological discoveries and medical treatments. The selective permittivity of the cell membrane is necessary for the survival of the cell; thus, all intracellular delivery methods must allow only the molecules of interest to pass through the cell membrane or the permeabilization of the membrane must be brief and reversible. The importance of intracellular delivery has given rise to numerous methods over the years that have been classified by their delivery mechanisms or by the quality and throughput of delivery resulting from these mechanisms. Reversible electroporation is one of the most used methods for intracellular delivery alongside viral and chemical vectors. Electroporation involves subjecting cells to an electric field to induce permeabilization of the plasma membrane. This permeabilization is temporary and allows the delivery or extraction of molecules of interest such as drugs, proteins, and nucleic acids. Until now, most electroporation applications have relied on one form of electroporation known as bulk electroporation, where hundreds of thousands or millions of cells are placed within a cuvette and simultaneously electroporated. Bulk electroporation is effective at delivering to a large quantity of cells, but the coarseness of the method yields significant variations in delivery, resulting in necrosis of some cells and minimal delivery to others.

### BRIEF SUMMARY

The present embodiments provide porous substrate electroporation (PSEP) devices and methods. PSEP is an electroporation methodology that maintains the high throughput capability of bulk electroporation while enabling greater control over the delivery process. In PSEP, cells are cultured on porous substrates. During electroporation, only the regions of the cell over micro- or nanochannels in the substrate are exposed to the electric field and undergo permeabilization. The total area of the cell exposed to the electric field can be far below 1%, allowing for much less toxicity while still enabling delivery.

According to an embodiment, an electroporation device is provided that includes a plurality of independently controllable or addressable electrode pairs, a plurality of reaction chambers, each reaction chamber of the plurality of reaction chambers including a first chamber, a second chamber and a porous substrate separating the first chamber from the second chamber, each of the plurality of reaction chambers being disposed between one of the plurality of independently controllable or addressable electrode pairs, a plurality of first microfluidic channels configured to deliver a cargo solution from a cargo inlet port to the plurality of first chambers, and a plurality of second microfluidic channels configured to deliver a cell culture from a cell inlet port to the plurality of second chambers. In operation, application of a voltage to an electrode pair permeabilizes the membranes of the cells adhered to the porous substrate in the reaction chamber disposed between the electrode pair.

In certain aspects, the electrodes are replaceable or removable.

According to certain aspects, the plurality of first microfluidic channels are further configured to expel cargo solution from the plurality of first chambers to a cargo outlet port, and wherein the plurality of second microfluidic channels are further configured to expel cell culture from the plurality of second chambers to a cell outlet port.

According to certain aspects, application of the voltage causes the cargo solution to pass from the first chamber through the porous substrate and into the cells in the second chamber.

According to certain aspects, each porous substrate is a polymer membrane. According to certain aspects, at least one of the porous substrates is a polymer membrane.

According to certain aspects, a removable fixture is provided that is configured to enclose the electroporation device, the removable fixture including a plurality of electrical ports enabling connection of the plurality of independently controllable or addressable electrode pairs to a voltage generator device or a multimeter device.

According to certain aspects, the electroporation device includes a first printed circuit board (PCB) and a second PCB, wherein the first PCB includes a first half of the plurality of independently controllable or addressable electrode pairs and wherein the second PCB includes a complementary half of the plurality of independently controllable or addressable electrode pairs. In certain aspects, the first PCB is attached to the plurality of first microfluidic channels and the second PCB is attached to the plurality of second microfluidic channels. In certain aspects, the first PCB includes a first electrical port enabling connection of the first half of the plurality of electrodes to a voltage generator device or a multimeter device, and the second PCB includes a second electrical port enabling connection of the complementary half of the plurality of electrodes to the voltage generator device or the multimeter device. In certain aspects, the first PCB includes a first plurality of O-rings and the second PCB includes a complementary plurality of O-rings arranged to mate with the first plurality of O-rings to secure the plurality of reaction chambers therebetween.

According to an embodiment, a method of delivering a cargo solution to a cell culture using the electroporation device is provided. The method may include applying voltage to one or more selected electrode pairs to cause cargo solution to pass from a cargo reservoir through a cargo inlet along microfluidic channels to one or more corresponding reaction chambers including the cell culture. Application of external peristaltic or syringe pumps may be applied to control flow of cargo out of one or more reaction chambers to a cargo outlet, as well as control ingress and egress of cell culture into and out of a one or more reaction chamber via the various microchannels.

According to an embodiment, a method of fabricating a porous substrate electroporation (PSEP) device is provided. The method includes forming a first mold assembly defining a first plurality of channels, a first plurality of chambers, a second plurality of channels and a second plurality of chambers, forming a second mold assembly defining a plurality of electrode pair locations, and injecting a polymer material into each of the first and second mold assemblies to form first and second molded parts, respectively, the first molded part including the first plurality of channels, the first plurality of chambers, the second plurality of channels and the second plurality of chambers, and the second molded part including the plurality of electrode locations. The method further includes separating the first and second molded parts from the first and second assemblies, inserting electrodes into the plurality of electrode pair locations on the second molded part, and coupling the first molded part with the second molded part to form an electroporation device structure including a plurality of reaction chambers, each reaction chamber of the plurality of reaction chambers including a first chamber, a second chamber and a porous substrate separating the first chamber from the second chamber, each of the plurality of reaction chambers being disposed between a pair of electrode pair locations.

According to another embodiment, an alternate method of fabricating a porous substrate electroporation (PSEP) device is provided. The method includes forming a first mold assembly defining a first plurality of channels and a first plurality of chambers, forming a second mold assembly defining a second plurality of channels and a second plurality of chambers, and injecting a polymer material into each of the first and second mold assemblies to form first and second molded parts, respectively, the first molded part including the first plurality of channels and the first plurality of chambers, and the second molded part including the second plurality of channels and the second plurality of chambers. The method also includes separating the first and second molded parts from the first and second assemblies, attaching a first printed circuit board (PCB) to the first molded part, wherein the first PCB includes a first half of a plurality of independently controllable or addressable electrode pairs, and attaching a second circuit board (PCB) to the second molded part, wherein the second PCB includes a complementary half of the plurality of independently controllable or addressable electrode pairs. The method further includes coupling the first molded part and first PCB with the second molded part and second PCB to form an electroporation device structure including a plurality of reaction chambers, each reaction chamber of the plurality of reaction chambers including a first chamber, a second chamber and a porous substrate separating the first chamber from the second chamber, each of the plurality of reaction chambers being disposed between one of the plurality of independently controllable or addressable electrode pairs.

In certain aspects, the first PCB includes a first electrical port enabling connection of the first half of the plurality of electrodes to a voltage generator device or a multimeter device, and the second PCB includes a second electrical port enabling connection of the complementary half of the plurality of electrodes to the voltage generator device or the multimeter device. In certain aspects, the first PCB includes, or is adapted to receive, a first plurality of O-rings and the second PCB includes, or is adapted to receive, a complementary plurality of O-rings arranged to mate with the first plurality of O-rings to secure the plurality of reaction chambers therebetween.

Reference to the remaining portions of the specification, including the drawings and claims, will realize other features and advantages of the present invention. Further features and advantages of the present invention, as well as the structure and operation of various embodiments of the present invention, are described in detail below with respect to the accompanying drawings. In the drawings, like reference numbers indicate identical or functionally similar elements.

### BRIEF DESCRIPTION OF THE DRAWINGS

FIG. 1 illustrates an isometric view of an electroporation device without a fixture, according to an embodiment.

FIG. 2 illustrates an isometric view of an electroporation a device with a fixture, according to an embodiment.

FIG. 3 shows a side view of an electroporation device, including operational features, according to an embodiment.

FIG. 4 shows a top view of an electroporation device, including microfluidic channels of the electroporation device, according to an embodiment.

FIG. 5 illustrates a side view of an electroporation a device with a fixture, according to an embodiment.

FIGS. 6A-6B show an example method for fabricating an electroporation device using injection molding, and attaching an external fixture thereto, according to an embodiment

FIG. 7 illustrates a prototype of an electroporation a device with a fixture, according to an embodiment.

FIG. 8 illustrates an electroporation a device, according to an embodiment: panel A shows a simplified depiction; panel B shows an enlarged view of cells on the membrane; panel C illustrates the reproducible device; and panel D shows an assembled device with a fixture.

FIG. 9 shows preliminary intracellular protein delivery data: the percentage of living cells (viability), the percentage of delivered cells (total delivery) and the percentage of cells that were delivered to and survived (delivery efficiency). The stars indicate the degree of statistical significance.

FIG. 10 shows representative images of intracellular protein delivery.

FIG. 11 shows a 3-D top perspective view of an electroporation device including a dual PCB assembly according to an embodiment.

FIG. 12 shows a top view of the electroporation device of FIG. 11.

FIG. 13 shows a 3-D bottom perspective view of the electroporation device of FIG. 11.

FIG. 14 shows a bottom view of the electroporation device of FIG. 11.

FIG. 15 shows a 3-D top perspective view of the electroporation device of FIG. 11 with the top PCB removed to show internal components.

FIG. 16 shows a top view of the electroporation device of FIG. 11 with the top PCB removed to show internal components.

FIG. 17 shows a cross-sectional view of an unsealed reaction chamber (cell culture chamber plus cargo chamber) according to an embodiment.

FIG. 18 shows a cross-sectional view of a sealed reaction chamber (cell culture chamber plus cargo chamber) according to an embodiment

### DETAILED DESCRIPTION

The present disclosure provides a mass-producible, standardized, and easy-to-use PSEP device and method for conducting porous substrate electroporation.

For existing PSEP systems, despite the many studies that have utilized PSEP for delivery, most studies have focused on biological applications and the underlying delivery mechanism remains relatively unexplored. Many PSEP parameters remain poorly understood because performing PSEP remains time-consuming and labor-intensive. PSEP often requires that new devices be made for each experiment, due to the difficulty in ensuring the narrow channels are free of previously used cargos and biological materials. Current PSEP devices require either cleanroom fabrication methods or require a separate device to be made for each sample

The present embodiments offer a number of advantages over existing PSEP systems that will enable PSEP device embodiments to serve as a high-throughput PSEP platform, including injection molding, miniature disposable electrodes, independent electrodes for each cell culture chamber, microfluidic channels, and reusable chambers. The present embodiments provide a novel injection mold design that allows the creation of two sets of microfluidic channels and chambers and the sealing of the porous membrane in a single step. The present embodiments also provide a design including formation of two separate molded parts and then sealing a membrane therebetween. The use of injection molding advantageously allows the present devices to be highly scalable and contain extremely consistent dimensions from device to device.

As for electrodes, devices may use a variety of materials including indium tin oxide coated glass, gold, stainless steel, silver-silver chloride, and titanium. Depending on the electrical parameters used, it may be important to replace electrodes between uses to minimize the effect of corrosion caused by the electric field. This corrosion significantly influences the electrical properties of the system and the voltage applied to the cells. The present devices utilize miniature, mass produced electrodes that can be easily replaced and only cost a few cents. Furthermore, the present devices are the first to contain independently controllable electrodes. Other devices require that the same electrical parameters be applied to all samples, preventing different electrical parameters from being assessed on the same device. The present devices separate these electrodes so that each sample is isolated from the influence of the other electrodes and each electrode has its own port for connecting to a voltage generator or multimeter. Microfluidic channels are used in the present devices because they allow fluidic handling steps to be highly scalable, allowing the transport of cell culture media and cargo solutions to be controlled simultaneously across numerous samples.

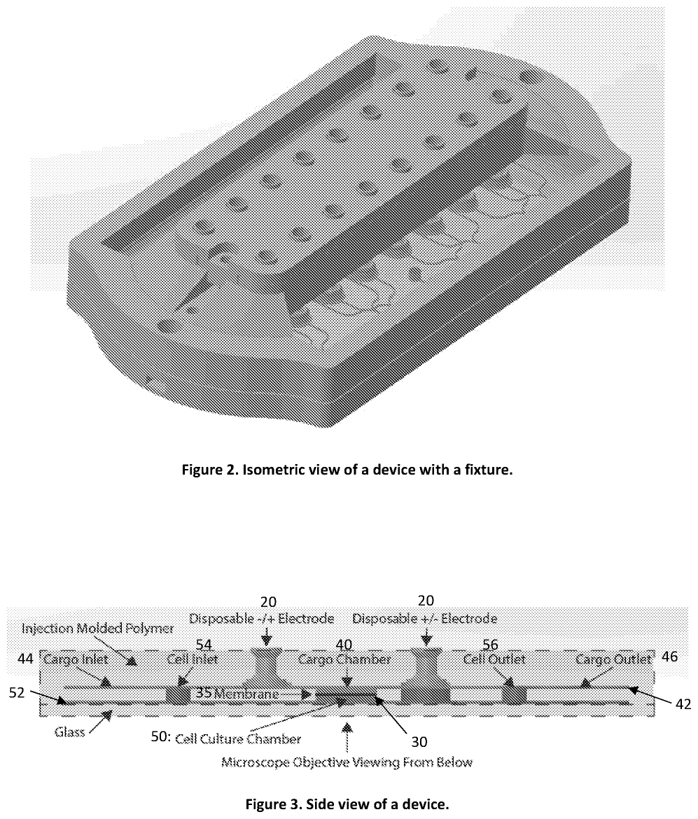

In addition to the aforementioned features that allow the present devices to be highly reproducible and scalable, the present devices are designed to be robust and easy to use. Other PSEP devices require the user to delicately position electrodes or carefully pipette liquids into small chambers. These processes lead to inconsistent results and make the device difficult to use by those who are unfamiliar with it. In certain embodiments, devices are designed with an attachable fixture (see, e.g., FIG. 2) containing industry-standard electrode ports, ensuring consistent electrode placement and reliable electrical connections. In other embodiments, unique PCBs are used to electrically couple the separate electrodes with control components. The microfluidic channels in the present devices enable them to be used without being proficient at pipetting.

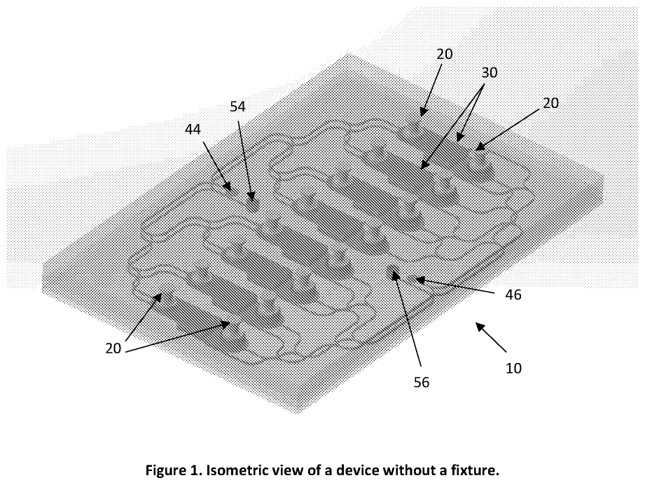

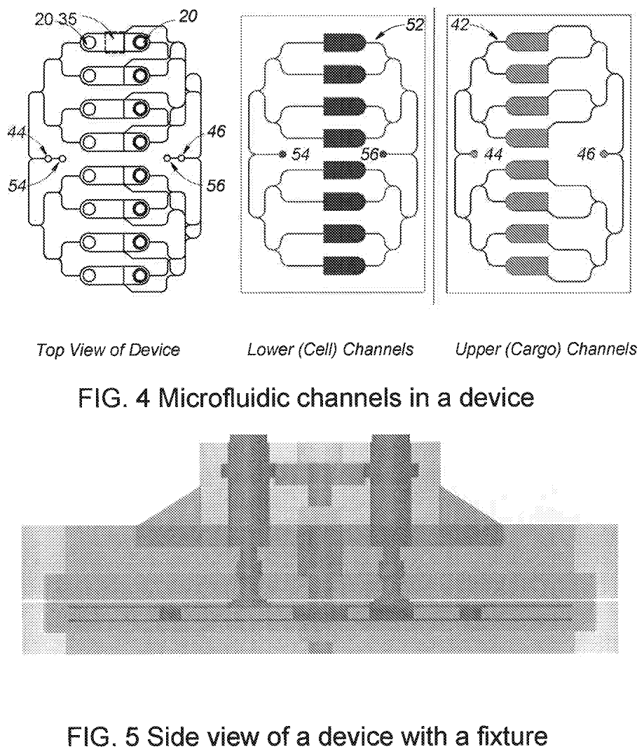

FIG. 1, FIG. 3 and FIG. 4 illustrate an isometric view of an electroporation device 10, a side view of the electroporation device 10, including operational features, and a top view of the electroporation device 10, including microfluidic channels of the electroporation device, respectively, according to an embodiment. Electroporation device 10 includes a plurality of independently controllable or addressable electrode pairs 20, and a plurality of reaction chambers 30 disposed therebetween. Each reaction chamber 30 includes a cargo chamber 40, a cell culture chamber 50 and a porous substrate (or membrane) 35 separating the cargo chamber 40 from the cell culture chamber 50. Each reaction chamber 30 is disposed between one of the plurality of independently controllable or addressable electrode pairs 20. Device 10 further includes a plurality of first microfluidic channels 42 configured to deliver a cargo solution from a cargo inlet port 44 to the plurality of cargo chambers 40, and a plurality of second microfluidic channels 52 configured to deliver cells and/or cell culture media from a cell inlet port 54 to the plurality of cell culture chambers 50. In operation, application of a voltage to an electrode pair 20 permeabilizes membranes of cells adhered to the porous substrate 35 in the reaction chamber 30 disposed between the activated electrode pair 20.

### Function

With reference to FIG. 3, the operation of a device according to an embodiment is as follows. Cells are added through the cell inlet port 54 and allowed to adhere to the porous substrate or membrane 35 in the cell culture chamber 50. The cargo solution for delivery is added through the cargo inlet port 44 to the cargo chamber 40. A voltage is then applied between the electrodes 20 to deliver cargo through the porous substrate 35 and into the cells, e.g., application of the voltage causes the cargo solution to pass from the cargo chamber through the porous substrate and into the cells in the cell culture chamber. After delivery, excess cargo is collected and/or expelled through the cargo outlet port 46. Cell culture media can be continuously replaced using the cell inlet port 54 and cell outlet port 56. The entire process can be imaged in real-time using a microscope objective from below. This advantageously enables dynamic modification of electrical parameters based on observed delivery.

According to an embodiment, each porous substrate includes a polymer membrane. Example polymers include polycarbonate, polyester, polyimide, and polydimethylsiloxane (PDMS).

In an embodiment, the two layers of microfluidic channels 42, 52 are produced simultaneously using extruded features on the upper and lower surfaces of a mold as shown in FIG. 4. The channels are designed for symmetric fluid flow and minimal electrical influence between samples. In the example embodiment shown, since all 8 samples are electrically connected by their common microfluidic channels, finite element analysis was used to estimate the voltage generated from one sample on the others (not shown here). The simulations suggest the voltage generated across non-targeted samples is less than 1% of the voltage generated across the targeted sample.

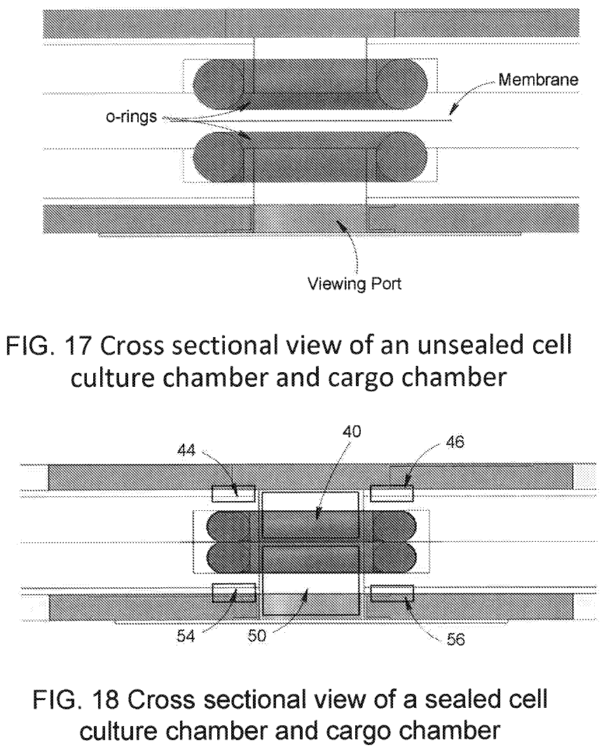

In an embodiment, during fabrication, membrane 35 material may be placed in the mold prior to filling the mold with liquid polymer. After curing of the liquid polymer, the membranes 35 are sealed and permanently embedded in the electroporation device 10. In another embodiment, the electroporation device 10 may be injection molded as 2 separate parts without the membrane 35, and then the membrane 35 is sealed within the device between 2 sets of O-rings (see, e.g., FIGS. 17-18). For example, each complimentary O-ring may surround one of the cargo chambers 40 and the other O-ring may surround the corresponding cell culture chamber 50. After each use, the O-rings may be separated and the membrane 35 can be removed and replaced and the device may be reused.

In an embodiment, a fixture is added around the device during operation to provide electrical ports for easy interface with the voltage generator. Unlike the electrodes, these ports can be reused numerous times since they do not contact the electrolyte solutions. FIG. 2 and FIG. 5 illustrate an isometric view and a side view, respectively, of electroporation device 10 with a fixture, according to an embodiment. The removable fixture is configured to enclose the electroporation device 10, the removable fixture including a plurality of electrical ports enabling connection of the plurality of independently controllable or addressable electrodes 20 to a voltage generator device or a multimeter device.

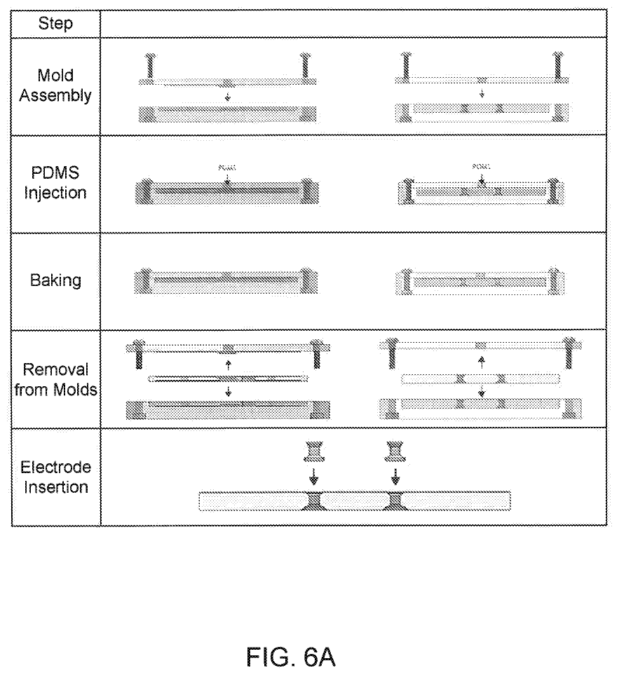

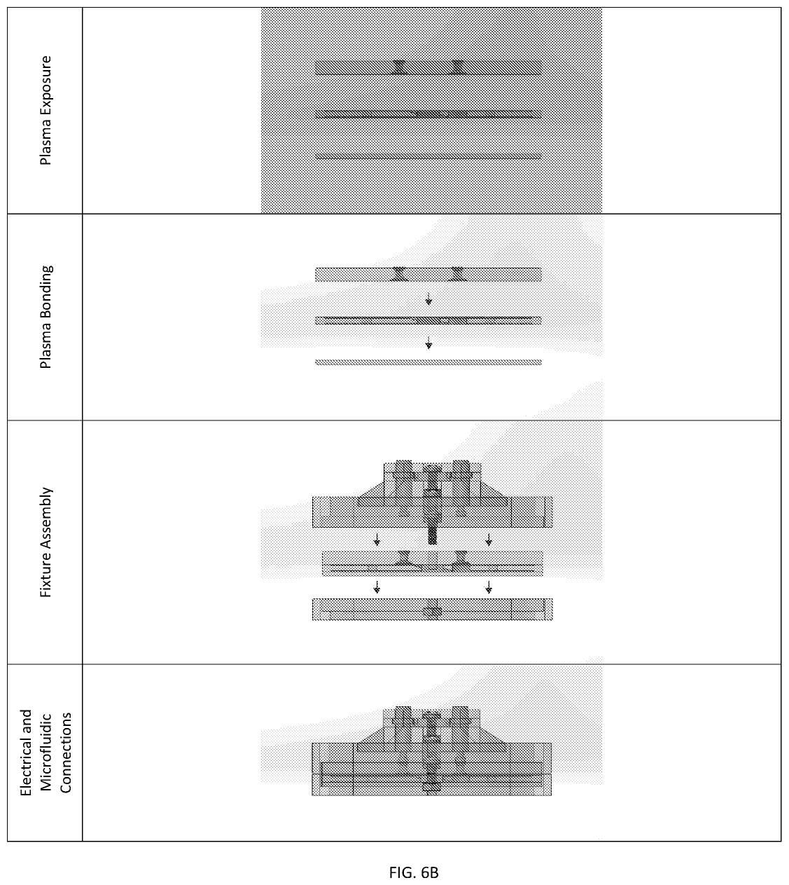

FIGS. 6A-6B show an example method for fabricating an electroporation device 10 using injection molding, and attaching an external fixture thereto, according to an embodiment. In a mold assembly step, a porous membrane material is placed between the two halves of the mold for the microfluidic channel layer. A second mold assembly is used to create the electrode layer. The two halves of the first mold are secured or clamped together, and the two halves of the second mold are secured or clamped together. In an injection step, a liquid polymer, such as PDMS or other useful polymer material, is injected into both molds using designated fill holes. Next, in a baking step, the molds are heated or baked, e.g., at 80° C. for an hour, to crosslink the polymer (e.g., PDMS). In a next step, the molds are unsecured or unclamped and the polymer is removed. Electrodes are then inserted into the electrode layer. In a plasma exposure step, the two polymer layers and a glass slide, or other substrate material, are exposed to air plasma or oxygen plasma. In a plasma bonding step, the lower face of the microfluidic channel layer is pressed against the glass slide to bond the surfaces together and the lower face of the electrode layer is pressed against the upper face of the microfluidic channel layer and bonded together. In a fixture assembly step, a fixture is clamped around the bonded polymer and glass slide to form an enclosed electroporation device. Thereafter, the electrodes and microfluidic channels (via the inlets and outlets) are connected to wires and tubing to interface with control circuitry and cell culture and cargo reservoirs. In operation, the same or different drive signals or waveforms may be applied to each separate electrode or electrode pair, to separately control each chamber 30 as desired.

The present embodiments also enable one to perform a novel method of PSEP involving long-term (e.g., >24 hours) delivery. To date, PSEP has only been demonstrated with the pulses applied over the period of a few seconds. It has been observed by the current inventors that the delivery efficiency is highest when shorter pulses and a greater number of pulses are used, thus elongating the period of time that the energy is applied to the cells. This is likely because continuous application results in the creation of too large of pores in the cell membrane or because of toxic byproducts that are produced at the electrode-electrolyte interface. It has also been theorized that the electric field reduces the seal between the cell membrane and the substrate over time, meaning less voltage is applied to the cell membrane. Therefore, it is expected that spacing out the pulses over minutes or hours, rather than seconds, will enable higher viability and delivery efficiency. PSEP is capable of performing long-term delivery because the cells are at their natural adherent state rather than in suspension as occurs during bulk electroporation. In addition, the present device embodiments may be designed to operate within an incubator and the microfluidic channels enable consistent replacement of cell culture media and the removal of toxic byproducts.

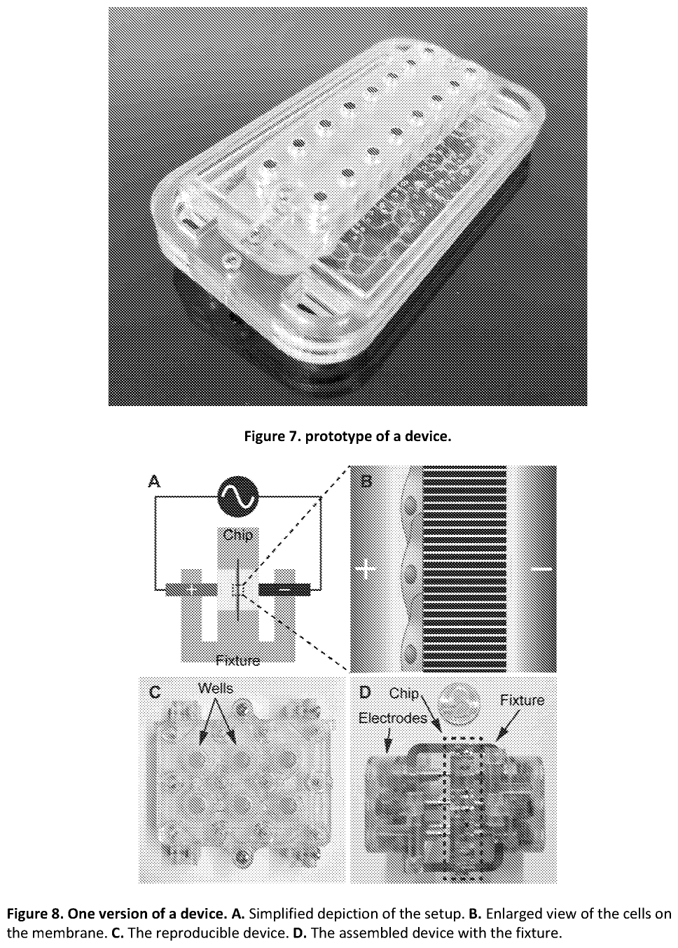

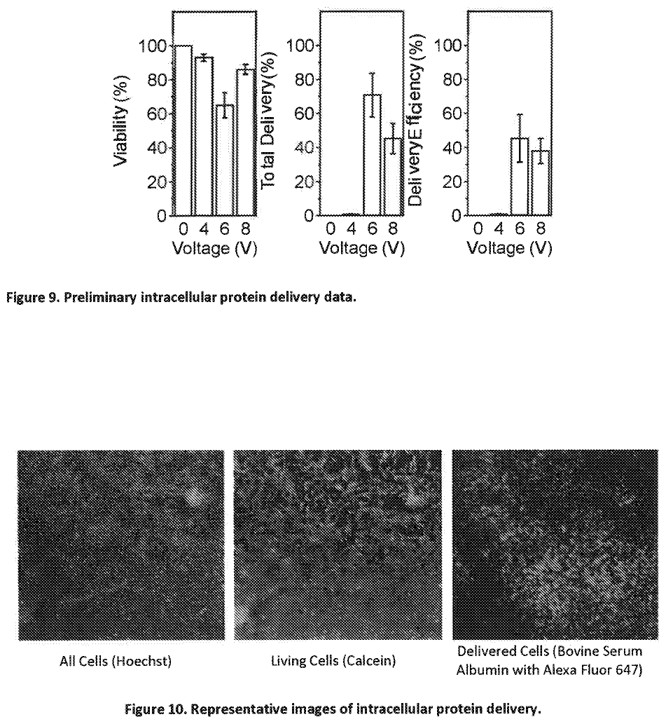

Preliminary data has been gathered for an example device's function and multiple prototypes have been produced. FIG. 7 shows an illustration of a device prototype according to an embodiment. In FIG. 8 (showing a simplified device), the device shown is horizontally oriented and does not contain microfluidic channels. The device's functionality has been demonstrated by delivering fluorescently tagged bovine serum albumin (BSA) into human embryonic kidney cells (HEK293) (see, FIG. 9). The protein was able to be delivered into most of the cells while keeping the cells alive. Representative images of the delivery are shown in FIG. 10.

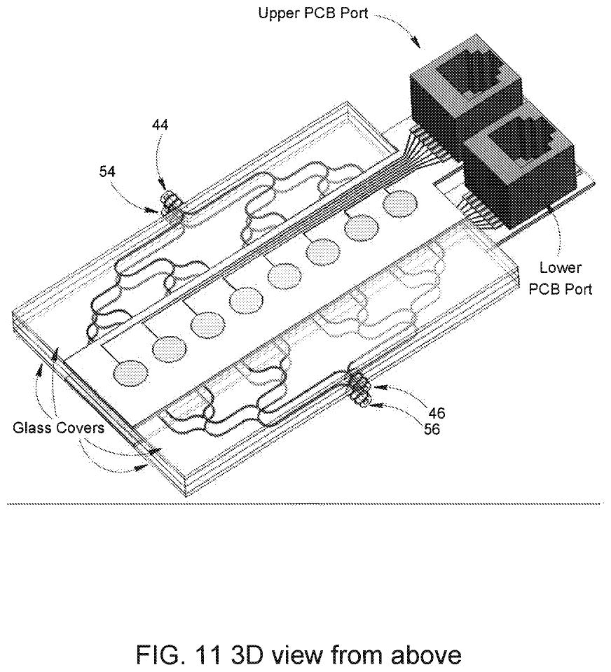

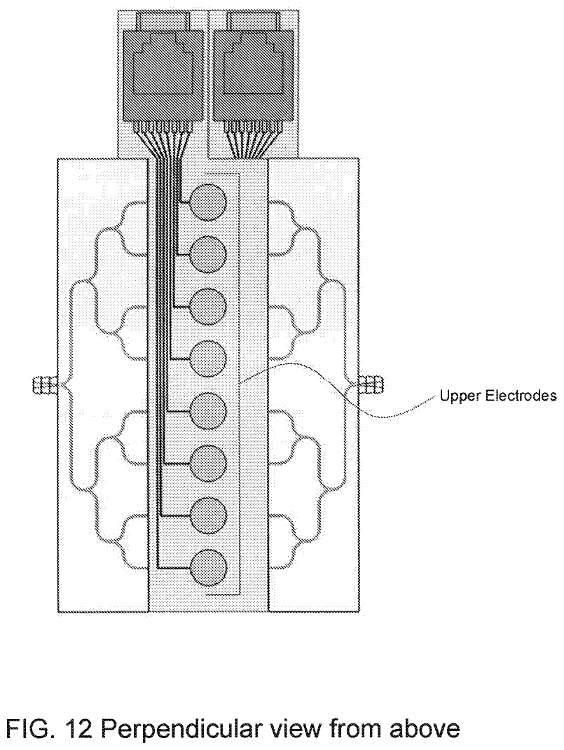

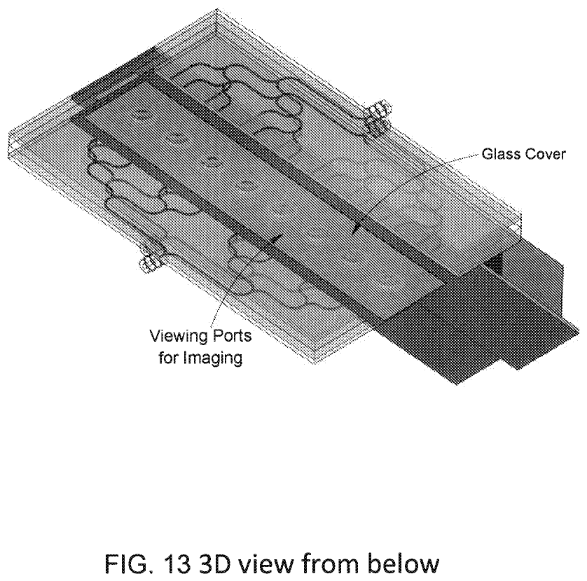

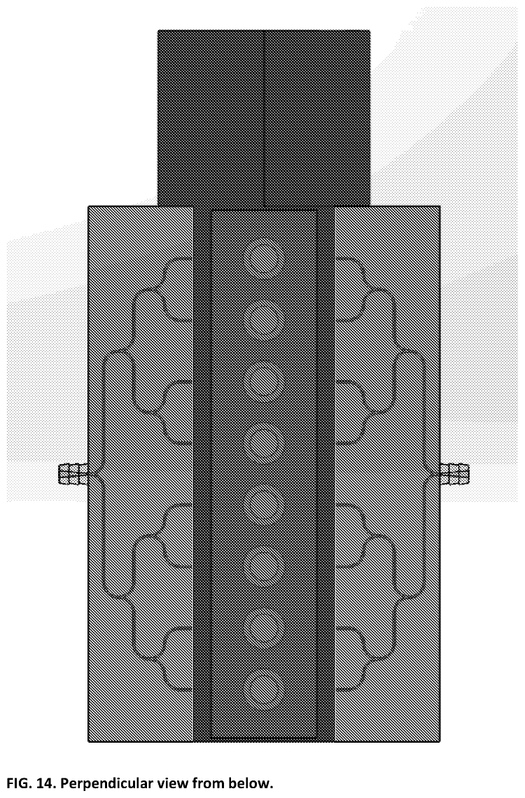

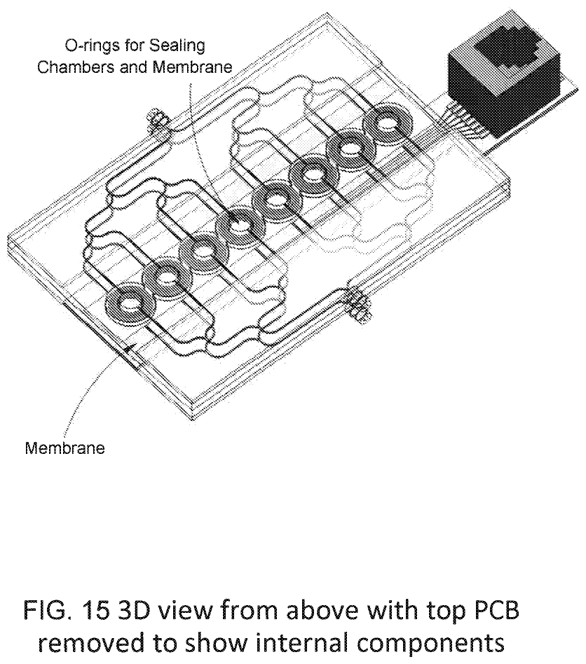

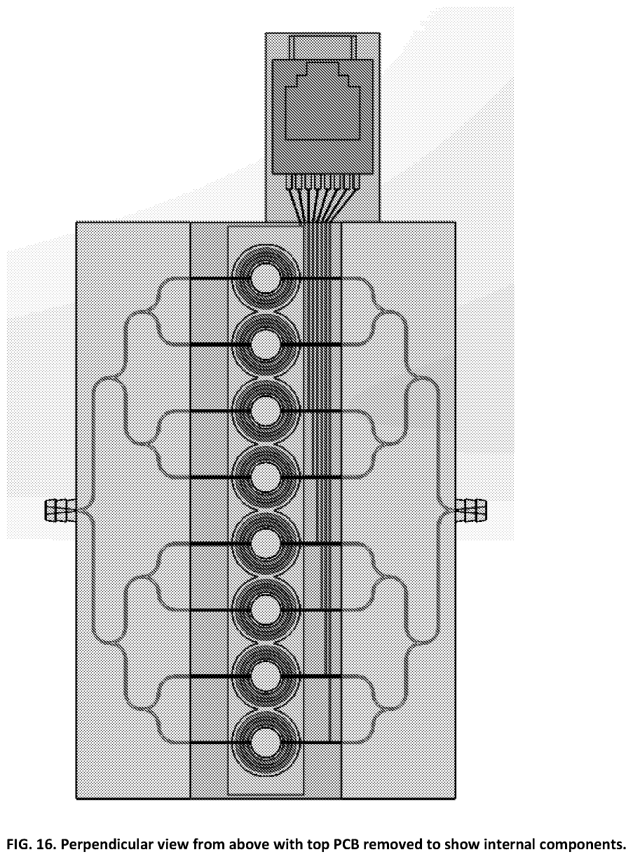

In another embodiment, the electrode fixture may be replaced with two printed circuit boards (PCBs) attached to two separate molds, one containing the cargo channels and the other containing the cell culture channels, as shown in FIGS. 11-16; one PCB contains the electrodes for the cell culture chambers 50 and a second PCB contains the electrodes for the cargo chambers 40. The PCBs may be permanently bonded to the injection molded channels for simplicity and robustness, but could also be sealed using O-rings similarly to the membranes as discussed above, allowing the electrodes to be replaced after they corrode. PCBs allow the electrodes to be placed closer together and scale more cheaply and easily than a large fixture. Electrical ports mounted on the PCBs allow the PCBs to be easily connected and disconnected from a function generator or multimeter. The conductors on the PCB may be plated, e.g., gold plated, to increase corrosion resistance. The lower PCB may contain a viewing port covered with a thin piece of glass for live imaging of the cells.

Additional information regarding intracellular delivery and PSEP can be found in Appendix A of U.S. provisional application 63/246,421, which is hereby incorporated by reference in its entirety.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### RELEVANT PUBLICATIONS

Brooks, J., Minnick, G., Mukherjee, P., Jaberi, A., Chang, L., Espinosa, H. D., & Yang, R. (2020). High Throughput and Highly Controllable Methods for In Vitro Intracellular Delivery. Small, 16(51), 2004917.
Kang, W., Giraldo-Vela, J. P., Nathamgari, S. S. P., McGuire, T., McNaughton, R. L., Kessler, J. A., & Espinosa, H. D. (2014). Microfluidic device for stem cell differentiation and localized electroporation of postmitotic neurons. Lab on a Chip, 14(23), 4486-4495.
Mukherjee, P., Berns, E. J., Patino, C. A., Hakim Moully, E., Chang, L., Nathamgari, S. S. P., . . . & Espinosa, H. D. (2020). Temporal Sampling of Enzymes from Live Cells by Localized Electroporation and Quantification of Activity by SAMDI Mass Spectrometry. Small, 16(26), 2000584.
Mukherjee, P., Nathamgari, S. S. P., Kessler, J. A., & Espinosa, H. D. (2018). Combined numerical and experimental investigation of localized electroporation-based cell transfection and sampling. ACS nano, 12(12), 12118-12128.
Cao, Y., Ma, E., Cestellos-Blanco, S., Zhang, B., Qiu, R., Su, Y., . . . & Yang, P. (2019). Nontoxic nanopore electroporation for effective intracellular delivery of biological macromolecules. Proceedings of the National Academy of Sciences, 116(16), 7899-7904.
Dong, Z., Yan, S., Liu, B., Hao, Y., Lin, L., Chang, T., . . . & Chang, L. (2021). Single Living Cell Analysis Nanoplatform for High-Throughput Interrogation of Gene Mutation and Cellular Behavior. Nano Letters.
Dong, Z., Jiao, Y., Xie, B., Hao, Y., Wang, P., Liu, Y., . . . & Chang, L. (2020). On-chip multiplexed single-cell patterning and controllable intracellular delivery. Microsystems & Nanoengineering, 6(1), 1-11.
Brooks, J. R., Mungloo, I., Mirfendereski, S., Quint, J. P., Paul, D., Jaberi, A., . . . & Yang, R. (2022). An equivalent circuit model for localized electroporation on porous substrates. Biosensors and Bioelectronics.

1. An electroporation device, comprising:
a plurality of independently controllable or addressable electrode pairs;
a plurality of reaction chambers, each reaction chamber of the plurality of reaction chambers including a first chamber, a second chamber and a porous substrate separating the first chamber from the second chamber, each of the plurality of reaction chambers being disposed between one of the plurality of independently controllable or addressable electrode pairs;
a plurality of first microfluidic channels configured to deliver a cargo solution from a cargo inlet port to the plurality of first chambers; and
a plurality of second microfluidic channels configured to deliver a cell culture from a cell inlet port to the plurality of second chambers;
wherein application of a voltage to an electrode pair permeabilizes membranes of cells adhered to the porous substrate in the reaction chamber disposed between the electrode pair.

2. The electroporation device of claim 1, wherein the plurality of first microfluidic channels are further configured to expel cargo solution from the plurality of first chambers to a cargo outlet port, and wherein the plurality of second microfluidic channels are further configured to expel cell culture from the plurality of second chambers to a cell outlet port.

3. The electroporation device of claim 1, wherein application of the voltage causes the cargo solution to pass from the first chamber through the porous substrate and into the cells in the second chamber.

4. The electroporation device of claim 1, wherein each porous substrate is a polymer membrane.

5. The electroporation device of claim 1, further including a removable fixture configured to enclose the electroporation device, the removable fixture including a plurality of electrical ports enabling connection of the plurality of independently controllable or addressable electrode pairs to a voltage generator device or a multimeter device.

6. The electroporation device of claim 1, further including a first printed circuit board (PCB) and a second PCB, wherein the first PCB includes a first half of the plurality of independently controllable or addressable electrode pairs and wherein the second PCB includes a complementary half of the plurality of independently controllable or addressable electrode pairs.

7. The electroporation device of claim 6, wherein the first PCB is attached to the plurality of first microfluidic channels and the second PCB is attached to the plurality of second microfluidic channels.

8. The electroporation device of claim 6, wherein the first PCB includes a first electrical port enabling connection of the first half of the plurality of electrodes to a voltage generator device or a multimeter device, and wherein the second PCB includes a second electrical port enabling connection of the complementary half of the plurality of electrodes to the voltage generator device or the multimeter device.

9. The electroporation device of claim 6, wherein the first PCB includes a first plurality of O-rings and the second PCB includes a complementary plurality of O-rings arranged to mate with the first plurality of O-rings to secure the plurality of reaction chambers therebetween.

10. A method of manufacturing a porous substrate electroporation (PSEP) device, comprising:
forming a first mold assembly defining a first plurality of channels, a first plurality of chambers, a second plurality of channels and a second plurality of chambers;
forming a second mold assembly defining a plurality of electrode pair locations;
injecting a polymer material into each of the first and second mold assemblies to form first and second molded parts, respectively, the first molded part including the first plurality of channels, the first plurality of chambers, the second plurality of channels and the second plurality of chambers, and the second molded part including the plurality of electrode locations;
separating the first and second molded parts from the first and second mold assemblies;
inserting electrodes into the plurality of electrode pair locations on the second molded part;
coupling the first molded part with the second molded part to form an electroporation device structure including a plurality of reaction chambers, each reaction chamber of the plurality of reaction chambers including a first chamber, a second chamber and a porous substrate separating the first chamber from the second chamber, each of the plurality of reaction chambers being disposed between a pair of electrode pair locations.

11. The method of claim 10, wherein each of the plurality of first microfluidic channels are configured to expel cargo solution from the plurality of first chambers to a cargo outlet port, and wherein the plurality of second microfluidic channels are further configured to expel cell culture from the plurality of second chambers to a cell outlet port.

12. The method of claim 10, wherein the injected polymer material includes polydimethylsiloxane (PDMS), polycarbonate, or other biocompatible polymers.

13. The method of claim 10, further including attaching a removable fixture to the first molded part and the second molded part to enclose the electroporation device, the removable fixture including a plurality of electrical ports enabling connection of the plurality of independently controllable or addressable electrode pairs to a voltage generator device or a multimeter device.

14. A method of manufacturing a porous substrate electroporation (PSEP) device, comprising:
forming a first mold assembly defining a first plurality of channels and a first plurality of chambers;
forming a second mold assembly defining a second plurality of channels and a second plurality of chambers;
injecting a polymer material into each of the first and second mold assemblies to form first and second molded parts, respectively, the first molded part including the first plurality of channels and the first plurality of chambers, and the second molded part including the second plurality of channels and the second plurality of chambers;
separating the first and second molded parts from the first and second mold assemblies;
attaching a first printed circuit board (PCB) to the first molded part, wherein the first PCB includes a first half of a plurality of independently controllable or addressable electrode pairs;
attaching a second circuit board (PCB) to the second molded part, wherein the second PCB includes a complementary half of the plurality of independently controllable or addressable electrode pairs;
coupling the first molded part and first PCB with the second molded part and second PCB to form an electroporation device structure including a plurality of reaction chambers, each reaction chamber of the plurality of reaction chambers including a first chamber, a second chamber and a porous substrate separating the first chamber from the second chamber, each of the plurality of reaction chambers being disposed between one of the plurality of independently controllable or addressable electrode pairs.

15. The method of claim 14, wherein the first PCB includes a first electrical port enabling connection of the first half of the plurality of electrodes to a voltage generator device or a multimeter device, and wherein the second PCB includes a second electrical port enabling connection of the complementary half of the plurality of electrodes to the voltage generator device or the multimeter device.

16. The method of claim 14, wherein the first PCB includes a first plurality of O-rings and the second PCB includes a complementary plurality of O-rings arranged to mate with the first plurality of O-rings to secure the plurality of reaction chambers therebetween.
